(19) United States
(12) Patent Application Publication
(10) Pub. No.: US 2008/0161875 A1
(43) Pub. Date: Jul. 3, 2008

(54) Gastric restriction method and system for treatment of eating disorders

(76) Inventor: Robert T. Stone, Sunnyvale, CA (US)

Correspondence Address: Ralph C. Francis, Francis Law Group, 1942 Embarcadero, Oakland, CA, 94606, US

(21) Appl. No.: 11/982,651

(22) Filed: Nov. 1, 2007

Related U.S. Application Data
(60) Provisional application No. 60/860,965, filed on Nov. 22, 2006.

(51) Int. Cl. A61N 1/36; A61B 17/08

(52) U.S. Cl. 607/40; 606/157

(57) ABSTRACT

A method for treating eating disorders comprising the steps of generating a neuro-electrical satiety signal that substantially corresponds to a neuro-electrical signal that is generated in a body and produces a satiety effect in the body, constricting the stoma of the stomach, and transmitting the neuro-electrical satiety signal to the subject.

### CROSS-REFERENCE TO RELATED APPLICATIONS

This application claims the benefit of U.S. Provisional Application No. 60/860,965, filed Nov. 22, 2006.

### FIELD OF THE PRESENT INVENTION

The present invention relates generally to medical methods and systems for treating eating disorders. More particularly, the invention relates to a gastric restriction method and system for treatment of eating disorders that includes means for generating and transmitting synthesized neurosignals that substantially correspond to neuro-electrical signals that are generated in the body and produce a satiety effect in the body.

### BACKGROUND OF THE INVENTION

The increasing prevalence of eating disorders, particularly obesity, in adults (and children) is one of the most serious and widespread health problems facing the world community. It is estimated that currently in America 55% of adults are obese and 20% of teenagers are either obese or significantly overweight. Additionally, 6% of the total population of the United States is morbidly obese.

This data is alarming for numerous reasons, not the least of which is it indicates an obesity epidemic. Many health experts believe that obesity is the first or second leading cause of preventable deaths in the United States, with cigarette smoking either just lagging or leading.

It is the consequences of being overweight that are most alarming. Obesity is asserted to be the cause of approximately eighty percent of adult onset diabetes in the United States, and of ninety percent of sleep apnea cases. Obesity is also a substantial risk factor for coronary artery disease, stroke, chronic venous abnormalities, numerous orthopedic problems and esophageal reflux disease. More recently, researchers have documented a link between obesity, infertility and miscarriages, as well as post menopausal breast cancer.

Despite these statistics, treatment options for obese people are limited. Classical models combining nutritional counseling with exercise and education have not led to long term success for very many patients. Use of liquid diets and pharmaceutical agents may result in weight loss which, however, is only rarely sustained.

Surgical procedures that cause either gastric restriction or malabsorption have been, collectively, the most successful long-term remedy for severe obesity. There are, however, several drawbacks associated with gastric restriction systems. One drawback is that the systems typically cannot be modified readily as patient needs demand or change.

Various “electrical stimulation” apparatus, systems and methods have also been employed to treat compulsive overeating and obesity. The noted systems and methods typically include the transmission of a pre-programmed electrical pulse or signal to a subject to induce a satiety effect, e.g., feeling of fullness. Illustrative are the systems and methods disclosed in U.S. Pat. Nos. 5,263,480 and 6,587,719, and U.S. Pat. Application Publications 2005/0033376 A1 and 2004/0024428 A1.

A major drawback associated with the “electrical stimulation” systems and methods disclosed in the noted patents and publications, as well as most known systems, is that the stimulus signals that are generated and transmitted to a subject are “user determined” and, in many instances “device determinative” (e.g., neurostimulator). Since the “stimulus signals” are not related to or representative of the signals that are generated in the body, the stimulus levels required to achieve the desired satiety effect are often excessive and can elicit deleterious side effects.

It would thus be desirable to provide a gastric restriction method and system for treating eating disorders that includes means for generating and transmitting synthesized neurosignals to a subject's body that substantially correspond to neuro-electrical coded signals that are generated in the body and produce or induce a satiety effect in the body.

It is therefore an object of the present invention to provide a gastric restriction method and system for treating eating disorders that overcomes the drawbacks associated with prior art methods and systems for treating eating disorders.

It is another object of the invention to provide a gastric restriction method and system for treating eating disorders that includes means for recording neuro-electrical signals that are generated in the body and produce a satiety effect in the body.

It is another object of the invention to provide a gastric restriction method and system for treating eating disorders that includes means for generating synthesized neurosignals that substantially correspond to neuro-electrical signals that are generated in the body and produce a satiety effect in the body.

It is another object of the invention to provide a gastric restriction method and system for treating eating disorders that includes means for transmitting synthesized neurosignals to a subject's body that substantially correspond to neuro-electrical signals that are generated in the body and produce a satiety effect in the body.

It is another object of the invention to provide a gastric restriction method and system for treating eating disorders that includes means for constricting the stoma of the stomach and timed transmission of synthesized neurosignals to a subject's body that substantially correspond to neuro-electrical signals that are generated in the body and produce a satiety effect in the body.

It is another object of the invention to provide a gastric restriction method and system for treating eating disorders that includes means for constricting the stoma of the stomach manual transmission of synthesized neurosignals to a subject's body that substantially correspond to neuro-electrical signals that are generated in the body and produce a satiety effect in the body.

### SUMMARY OF THE INVENTION

In accordance with the above objects and those that will be mentioned and will become apparent below, in one embodiment of the invention, the gastric restriction method for treating eating disorders generally comprises includes the steps of (i) generating a synthesized neurosignal that substantially corresponds to a neuro-electrical signal that is generated in a body and produces a satiety effect in the body, (ii) constricting the stoma of the stomach, and (iii) transmitting the synthesized neurosignal to the subject.

In one embodiment, the synthesized neurosignal is transmitted at predetermined time intervals.

In one embodiment, the synthesized neurosignal is transmitted manually.

In another embodiment, the synthesized neurosignal is transmitted manually and at predetermined time intervals.

In one embodiment of the invention, the synthesized neurosignal has a first region having a first positive voltage in the range of approximately 100-1500 mV for a first period of time in the range of approximately 100-400 μsec and a second region having a first negative voltage in the range of approximately −50 mV to −750 mV for a second period of time in the range of approximately 200-800 μsec.

In a preferred embodiment of the invention, the first positive voltage is approximately 800 mV, the first period of time is approximately 200 μsec, the first negative voltage is approximately −400 mV and the second period of time is approximately 400 μsec.

Preferably, the synthesized neurosignals has a repetition rate in the range of approximately 0.01-4 KHz.

In another embodiment of the invention, the gastric restriction method for treating eating disorders includes the steps of (i) capturing neuro-electrical signals that are generated in the body and produce a satiety effect in the body, (ii) generating a synthesized neurosignal that substantially corresponds to at least one of the captured neuro-electrical signals, and (iii) transmitting the synthesized neurosignal to the subject.

In another embodiment of the invention, the gastric restriction method for treating eating disorders includes the steps of (i) capturing neuro-electrical signals that are generated in the body and produce a satiety effect in the body, (ii) generating a synthesized neurosignal that substantially corresponds to at least one of the captured neuro-electrical signals, (iii) constricting the stoma of the stomach, and (iv) transmitting the synthesized neurosignal to the subject.

The gastric restriction system for treating eating disorders, in accordance with one embodiment of the invention, generally comprises (i) a gastric band adapted to constrict the stoma of the stomach, (ii) a processor adapted to generate at least a first synthesized neurosignal that substantially corresponds to a neuro-electrical signal that is generated in the body and produces a satiety effect in the body, and (iii) a signal transmitter adapted to be in communication with the subject's body for transmitting the first synthesized neurosignal to the subject.

In one embodiment of the invention, the system includes a remote transponder that is adapted to transmit control signals to the processor.

In one embodiment of the invention, the remote transponder is further adapted to remotely monitor the control parameters of the processor.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1A is an illustration of a portion of a human torso, showing the gastrointestinal tract;

FIG. 1B is an illustration of a human stomach;

FIG. 2 is a schematic illustration of one embodiment of a synthesized neurosignal of the invention;

FIG. 3 is a schematic illustration of one embodiment of a food intake control system, according to the invention;

FIG. 4 is a schematic illustration of another embodiment of a food intake control system, according to the invention;

FIG. 5 is a schematic illustration of another embodiment of a food intake control system, according to the invention;

FIG. 6 is a perspective view of a prior art gastric band that is adapted to constrict the stoma of the stomach;

FIG. 7 is another perspective view of the gastric band shown in FIG. 6, illustrating an expanded state of the inflatable member;

FIG. 8 is a further illustration of the partial human torso shown in FIG. 1A, illustrating the placement of the gastric band shown in FIG. 6 on the neck region of the stomach;

FIG. 9 is a perspective view of one embodiment of a gastric restriction system, comprising the gastric band shown in FIG. 6 and one embodiment of the control system shown in FIG. 4, according to the invention;

FIGS. 10 and 11 are perspective views of additional embodiments of the gastric restriction system shown in FIG. 9, according to the invention;

FIG. 12 is a perspective view of yet another embodiment of the gastric restriction system shown in FIG. 9, according to the invention;

FIG. 13 is a perspective view of the gastric restriction system shown in FIG. 9, showing an expanded state of the inflatable member;

FIG. 14 is a further illustration of the partial human torso shown in FIG. 1, illustrating the restrictive placement of the gastric restriction system shown in FIG. 9 on the neck region of the stomach, according to the invention;

FIG. 15 is a further illustration of the partial human torso shown in FIG. 1, illustrating the “non-restrictive” placement of the gastric restriction system shown in FIG. 9 on the neck region of the stomach, according to the invention; and

FIG. 16 is a front plan view of one embodiment of a gastric electrode positioning device, according to the invention.

### DETAILED DESCRIPTION OF THE INVENTION

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred systems and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a synthesized signal” includes two or more such signals; reference to “a neuro-electrical signal” includes two or more such signals and the like.

### Definitions

The terms “patient” and “subject”, as used herein, mean and include humans and animals.

The terms “satiety” and “satiety effect”, as used herein, mean a feeling of fullness experienced by a subject.

The term “eating disorder”, as used herein, means and includes, without limitation, compulsive eating and obesity, bulimia and anorexia nervosa.

The term “nervous system”, as used herein, means and includes the central nervous system, including the spinal cord, medulla oblongata, pons, cerebellum, midbrain, diencephalon and cerebral hemisphere, and the peripheral nervous system, including the neurons and glia.

The term “plexus”, as used herein, means and includes a branching or tangle of nerve fibers outside the central nervous system.

The term “ganglion”, as used herein, means and includes a group or groups of nerve cell bodies located outside the central nervous system.

The terms “vagus nerve” and “vagus nerve bundle” are used interchangeably herein and mean and include one of the twelve (12) pair of cranial nerves that emanate from the medulla oblongata.

The term “neuro-electrical signal”, as used herein, means and includes a composite electrical signal that is generated in the body and carried by neurons in the body, including neurocodes, neurosignals and components and segments thereof, and generated neuro-electrical signals that substantially correspond thereto.

The term “synthesized neurosignal”, as used herein, means and includes an electrical signal made up of mathematical descriptors which, when applied to a animal nervous system elicit the same physiological response as a natural neuro-electrical signal elicits within the animal. In general, the amplitude and duration of synthesized neurosignals have key characteristics that are similar to action potentials found naturally in neurons; although their time-amplitude appearance may be different.

According to the invention, a synthesized neurosignal can be derived via various processes of synthesis, including, without limitation, time domain synthesis, frequency domain synthesis, bi-normal synthesis, and other conventional signal processing techniques.

The term “time domain synthesis”, as used herein, means synthesis or summation of components, which have an amplitude and duration as descriptive specifications of components of the signal which are linearly or non-linearly combined to form a composite signal.

The term “frequency domain synthesis”, as used herein, means synthesis or formation of a composite signal from elements that are specified as having a frequency, amplitude, and phase, which elements are combined either linearly or non-linearly to form the composite signal.

The term “synthesized satiety signal”, as used herein, means a synthesized neurosignal that produces or induces a satiety effect in a subject when transmitted thereto.

The term “digestion”, as used herein, means and includes all physiological processes associated with extracting nutrients from food and eliminating waste from the body.

The term “gastrointestinal system”, as used herein, means and includes, without limitation, the gastrointestinal tract and, hence, all organs and systems involved in the process of digestion. The “gastrointestinal system” also includes the nervous system associated with the noted organs and systems.

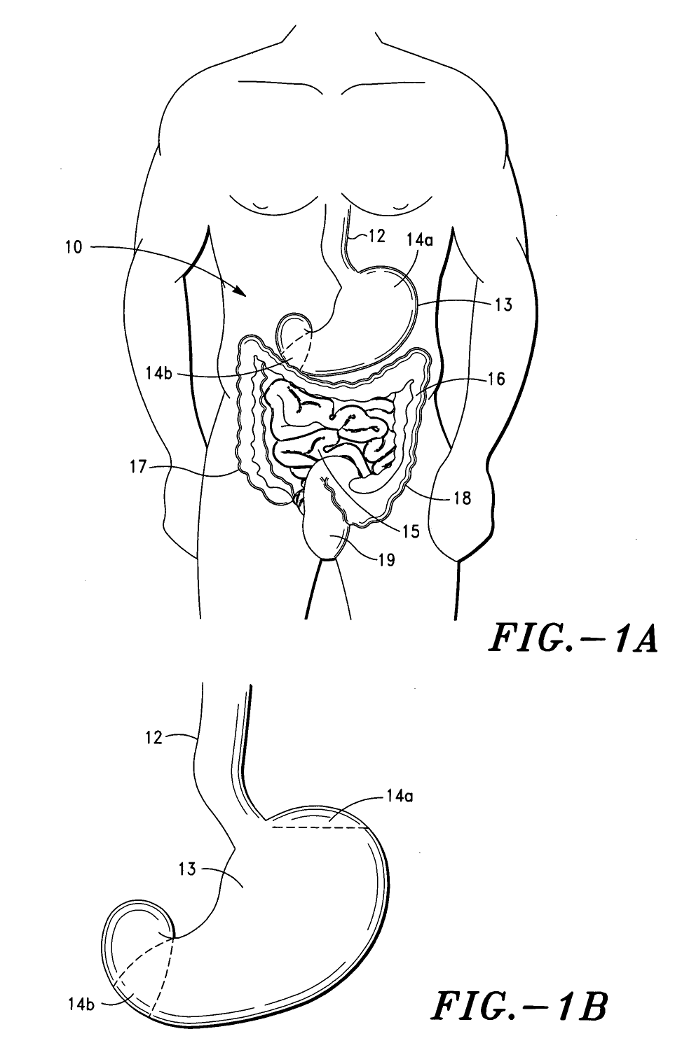

Referring first to FIG. 1A, there is shown an illustration of a typical gastrointestinal tract (designated generally “10”). As illustrated in FIG. 1, the gastrointestinal tract 10 generally includes the oesophagus or esophagus 12, stomach 13, small intestines 15 and large intestines 16, which includes the cecum 17, colon 18 and rectum 19. Referring to FIG. 1B, the stomach 13 includes the fundus region (or fundus) 14a and pyloric antrum (or antrum) 14b.

As is well known in the art, the brain regulates (or controls) feeding behavior and gastrointestinal function via electrical (or neuro-electrical) signals (i.e. action potentials), which are transmitted through the nervous system.

It is also well known in the art that an organism employs two main cues to regulate food intake; short term cues that regulate the size of individual meals and long-term cues that regulate overall body weight. Short-term cues consist primarily of chemical properties of the food that act in the mouth to stimulate feeding behavior and in the gastrointestinal system and liver to inhibit food intake. Short-term neuro-electrical (or satiety) signals, which are associated with (or provided by) the short-term cues, are transmitted through the nervous system and impinge on the hypothalamus through visceral afferent pathways, communicating primarily with the lateral hypothalamic regions (or satiety centers) of the brain.

The effectiveness of short-term cues is modulated by long-term neuro-electrical signals that reflect body weight. These long-term signals are similarly transmitted through the nervous system.

One important long-term neuro-electrical signal is the peptide leptin, which is secreted from fat storage cells (i.e. adipocytes). By means of this signal, body weight is kept reasonably constant over a broad range of activity and diet.

The neuro-electrical signals that are transmitted through the nervous system to regulate food intake and gastrointestinal function, referred to as action potentials, are rapid and transient “all-or-none” nerve impulses. Action potentials typically have an amplitude of approximately 100 millivolts (mV) and a duration of approximately 1 msec.

A “neurosignal”, as the term is commonly employed in the art, refers to a composite signal that includes many action potentials. The neurosignal also includes an instruction set for proper organ and/or system function. A neurosignal that controls gastrointestinal function would thus include an instruction set for the muscles of the colon and anus to perform an efficient elimination or retention of a stool bolus, including information regarding initial muscle tension, degree (or depth) of muscle movement, etc.

Neurosignals are thus signals that contain complete sets of information for control of organ function. As set forth in Co-Pending application Ser. No. 11/125,480, which is incorporated by reference herein, once these neurosignals have been isolated, recorded and processed, a nerve-specific signal or instruction, i.e. simulated neurosignal, can be generated and transmitted to a subject to control gastrointestinal function and, hence, treat a multitude of digestive system diseases and disorders, including, but not limited to, bowel (or fecal) incontinence, constipation and diarrhea.

As set forth in Co-Pending application Ser. Nos. 11/393,194, 11/431,869, which are incorporated by reference herein, a simulated neurosignal can also be generated and transmitted to a subject to regulate food intake and, hence, treat various eating disorders, including, but not limited to, compulsive overeating and obesity, bulimia and anorexia nervosa.

As discussed in detail in Co-pending U.S. application Ser. No. 11/134,767, which is incorporated by reference herein in its entirety, the vagus nerve bundle, which contains both afferent and efferent pathways, conducts neurosignals from the medulla oblongata to direct aspects of the digestive process, including the secretion of digestive chemicals, operation of the salivary glands and regulation of gastrointestinal muscles (e.g., puborectalis, puboccygeus and iliococcygeus muscles). The vagus nerve bundle thus plays a significant role in mediating afferent information from the stomach to the satiety centers of the brain.

As will be readily apparent to one having ordinary skill in the art, the present invention substantially reduces or eliminates the disadvantages and drawbacks associated with prior art systems and methods for treating eating disorders. As discussed in detail below, in accordance with some embodiments of the invention, the method for treating eating disorders includes the step of transmitting at least one synthesized, i.e. simulated, neurosignal to a subject that substantially corresponds to or is representative of at least one neuro-electrical signal that is naturally generated in the body and produces a satiety effect in the body. In one preferred embodiment, the synthesized neurosignal comprises a synthesized satiety signal that substantially corresponds to a short-term satiety signal that produces or induces a feeling a fullness.

Thus, in accordance with one embodiment of the invention, the method for treating eating disorders includes the steps of (i) generating at least one synthesized satiety signal that substantially corresponds to a neuro-electrical signal that is generated in a body and produces a satiety effect in the body and (ii) transmitting the synthesized satiety signal to the subject. In one embodiment, a plurality of synthesized satiety signals are generated and transmitted to the subject.

In some embodiments of the invention, the methods for treating eating disorders include the pre-programmed or timed transmission of the synthesized satiety signals. Thus, in the case of an obese or bulimic subject, a synthesized satiety signal or a plurality of synthesized satiety signals can be transmitted at set intervals at, near and/or between customary meal times to induce a feeling of fullness.

As discussed in detail herein, alternatively, or in addition to timed transmission of synthesized satiety signals, the transmission of the synthesized satiety signals can also be accomplished manually. As will be appreciated by one having skill in the art, manual transmission of a signal is useful in situations where the subject has an earnest desire to control his or her eating behavior, but requires supportive measures due to insufficient will power to refrain from compulsive and/or damaging behavior.

In yet further alternative embodiments of the invention, the methods for treating eating disorders includes the step of capturing neuro-electrical signals from a subject's body that produce a satiety effect in the body. According to the invention, the captured neuro-electrical signals can be employed to generate the synthesized neurosignals and/or synthesized satiety signals of the invention.

Methods and systems for capturing neuro-electrical signals from nerves, and for storing, processing and transmitting generated signals (or neurosignals) are set forth in Co-Pending U.S. patent application Ser. Nos. 11/125,480, filed May 9, 2005, 10/000,005, filed Nov. 20, 2001 and 11/147,497; which are incorporated by reference herein in their entirety.

According to the invention, suitable neuro-electrical signals that produce a satiety effect in the body can be captured or collected from the vagus nerve bundle. A preferred location is in the neck region of the stomach, which is enervated by the vagus nerve.

According to one embodiment of the invention, the captured neuro-electrical signals are preferably transmitted to a processor or control module. Preferably, the control module includes storage means adapted to store the captured signals. In a preferred embodiment, the control module is further adapted to store the components of the captured signals (that are extracted by the processor) in the storage means according to the function performed by the signal components.

According to the invention, the stored neuro-electrical signals can subsequently be employed to establish base-line satiety signals. The module can then be programmed to compare neuro-electrical signals (and components thereof) captured from a subject to base-line satiety signals and, in some embodiments, generate a synthesized neurosignal or satiety signal based on the comparison for transmission to a subject.

According to the invention, the captured neuro-electrical signals can be processed by known means to generate a synthesized neurosignal that produces a satiety effect in the body (i.e. a synthesized satiety signal). In a preferred embodiment, the synthesized neurosignal substantially corresponds to or is representative of at least one captured neuro-electrical signal. The synthesized neurosignal is similarly preferably stored in the storage means of the control module.

In one embodiment of the invention, the synthesized neurosignals are processed as follows: Sinusoidal elements of a frequency, amplitude, and phase, which have been identified from the recorded neuro-electrical signals as indicative of a sense of satiety or “fullness”, are combined into a composite signal. The resulting spectrum of the synthesized neurosignal (i.e. satiety signal) is transformed back into a time domain signal by a process known as Inverse Fourier Transform, and a digital representation of this signal is stored in a suitable neuro-signal generator for application to the desired nerve.

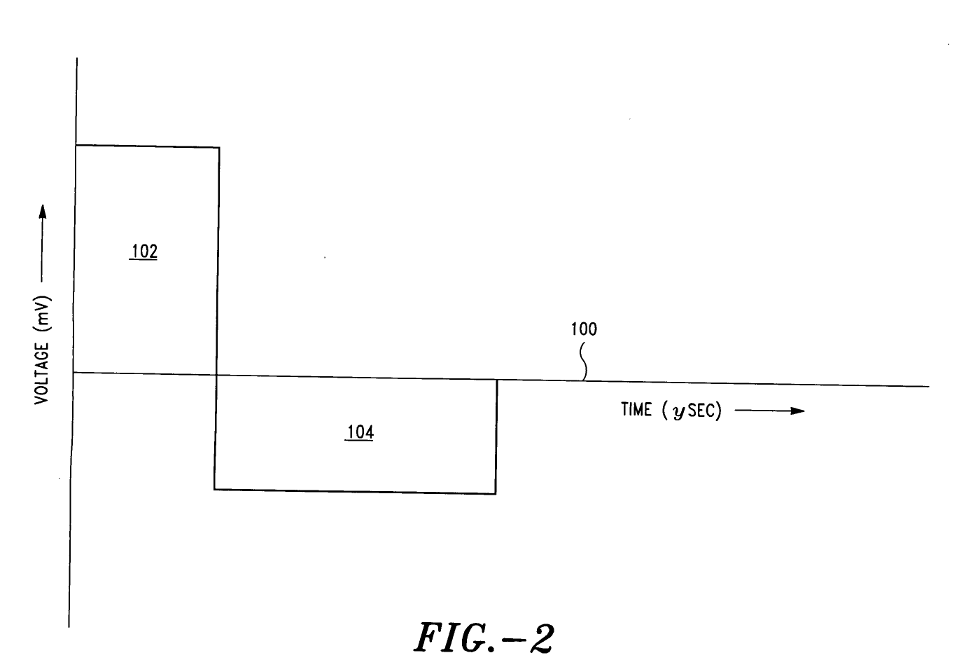

Referring now to FIG. 2, there is shown one embodiment of a synthesized neurosignal 100 of the invention, which has been derived via the frequency domain synthesis disclosed above. As indicated, the signal 100 substantially corresponds to or is representative of neuro-electrical signals that are naturally generated in the body and produce a satiety effect in the body. The signal 100 is thus referred to hereinafter as a “synthesized satiety signal”.

As illustrated in FIG. 2, the synthesized satiety signal 100 preferably includes a positive voltage region 102 having a first positive voltage (V₁) for a first period of time (T₁) and a first negative region 104 having a first negative voltage (V₂) for a second period of time (T₂).

Preferably, the first positive voltage (V₁) is in the range of approximately 100-1500 mV, more preferably, in the range of approximately 700-900 mV, even more preferably, approximately 800 mV; the first period of time (T₁) is in the range of approximately 100-400 μsec, more preferably, in the range of approximately 150-300 μsec, even more preferably, approximately 200 μsec; the first negative voltage (V₂) is in the range of approximately −50 mV to −750 mV, more preferably, in the range of approximately −350 mV to −450 mV, even more preferably, approximately −400 mV; the second period of time (T₂) is in the range of approximately 200-800 μsec, more preferably, in the range of approximately 300-600 μsec, even more preferably, approximately 400 μsec.

The synthesized satiety signal 100 thus comprises a continuous sequence of positive and negative voltage (or current) regions or bursts of positive and negative voltage (or current) regions, which preferably exhibits a DC component signal substantially equal to zero.

Preferably, the synthesized satiety signal 100 has a repetition rate (or frequency) in the range of approximately 0.01-4 KHz, more preferably, in the range of approximately 1-2 KHz. Even more preferably, the repetition rate is approximately 1.6 KHz.

According to the invention, the maximum amplitude of the synthesized satiety signal 100 is approximately 500 mV. In a preferred embodiment of the invention, the maximum amplitude of the synthesized satiety signal 100 is approximately 200 mV. As will be appreciated by one having ordinary skill in the art, the effective amplitude for the applied voltage is a strong function of several factors, including the electrode employed and the placement of the electrode(s).

According to the invention, the synthesized satiety signals of the invention can be employed to construct “signal trains”, comprising a plurality of synthesized satiety signals. The signal train can comprise a continuous train of synthesized satiety signals or can included interposed signals or rest periods, i.e., zero voltage and current, between one or more synthesized satiety signals.

The signal train can also comprise substantially similar synthesized satiety signals, different synthesized satiety signals or a combination thereof. According to the invention, the different synthesized satiety signals can have different first positive voltage (V₁) and/or first period of time (T₁) and/or first negative voltage (V₂) and/or second period of time (T₂).

In another embodiment of the invention, the synthesized satiety signal is derived via time domain synthesis. Details of a suitable time domain synthesis process and resulting synthesized neurosignals are set forth in Co-Pending application Ser. No. 11/265,402; which is incorporated herein in its entirety.

In response to a pre-programmed event, e.g., pre-programmed period of time or time interval or manual activation, the synthesized satiety signal (or signals) is accessed from the storage means and transmitted to the subject via a transmitter (or probe).

According to the invention, the applied voltage of the synthesized satiety signals of the invention can be up to 20 volts to allow for voltage loss during the transmission of the signals. Preferably, current is maintained to less than 2 amp output.

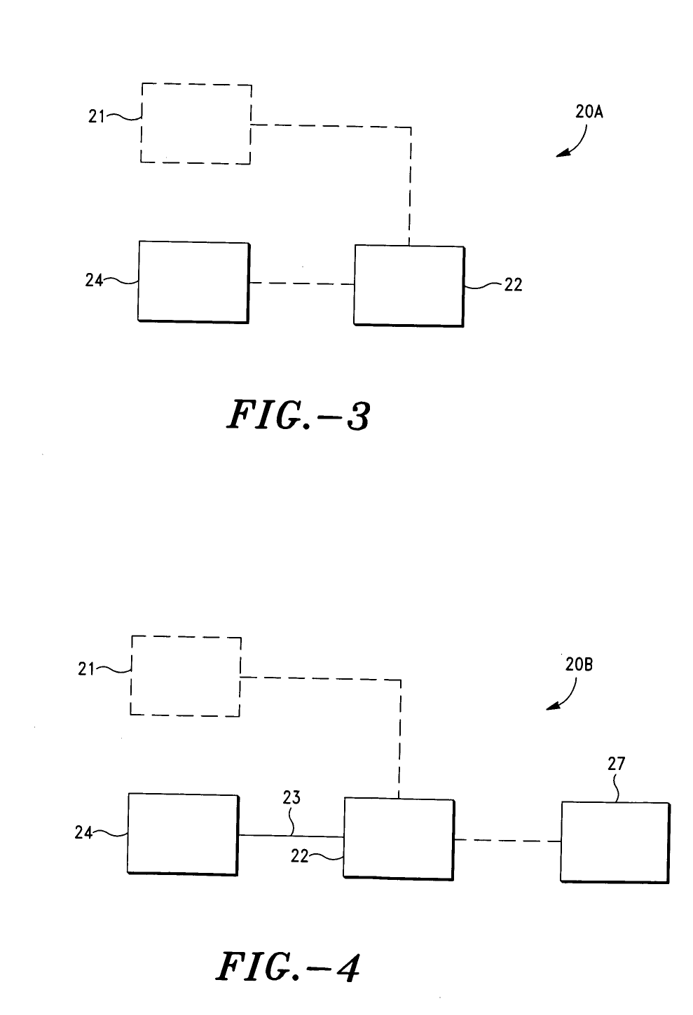

Referring now to FIG. 3, there is shown a schematic illustration of one embodiment of a food intake control system 20A of the invention. As illustrated in FIG. 3, the control system 20A includes a control module 22, which is adapted to receive neuro-electrical signals from a signal sensor (shown in phantom and designated 21) that is in communication with a subject, and at least one treatment member 24 that is adapted to communicate with the body.

The control module 22 is further adapted to generate synthesized satiety signals that substantially correspond to or are representative of neuro-electrical signals that are generated in the body and produce a satiety effect in the body, and transmit the synthesized satiety signals to the treatment member 24 at predetermined periods of time (or time intervals) and/or manually, i.e. upon activation of a manual switch (not shown).

According to the invention, the control module 22 can be unique, i.e., tailored to a specific operation and/or subject, or can comprise a conventional device.

As illustrated in FIG. 3, in one embodiment of the invention, the control module 22 and treatment member 24 are separate components or elements, which allows system 20A to be operated remotely. Thus, in some embodiments of the invention, such as the embodiment shown in FIG. 3, the module 22 is adapted to “wirelessly” transmit the synthesized satiety signals to the treatment member 24. As will be appreciated by one having ordinary skill in the art, various wireless transmission means can be employed within the scope of the invention to effectuate the wireless transmission of signals from the module 22 to the treatment member 24.

In other embodiments of the invention, the module 22 is adapted to transmit synthesized satiety signals to the treatment member 24 via at least one interconnect wire 23. Illustrative is the embodiment shown in FIG. 4, discussed below.

According to the invention, the treatment member 24 receives the synthesized satiety signals from the control module 22 and transmits the signals to the body. According to the invention, the treatment member 24 can comprise an electrode, antenna, a seismic transducer, or any other suitable form of conduction attachment for transmitting the neuro-electrical satiety signals to a subject. In one embodiment of the invention, discussed below, the treatment member 24 comprises an electrode system 25 (see FIG. 9).

Referring now to FIG. 4, there is shown a further embodiment of a control system 20B of the invention. As illustrated in FIG. 4, the system 20B is similar to system 20A shown in FIG. 3. However, in this embodiment, the control module 22 and treatment member 24 are connected via interconnect wire 23.

In one embodiment of the invention, the control system 20B includes a remote transponder 27 that is adapted to transmit control signals to the module 22. In another embodiment, the transponder 27 is further adapted to monitor the control parameters of the module 22.

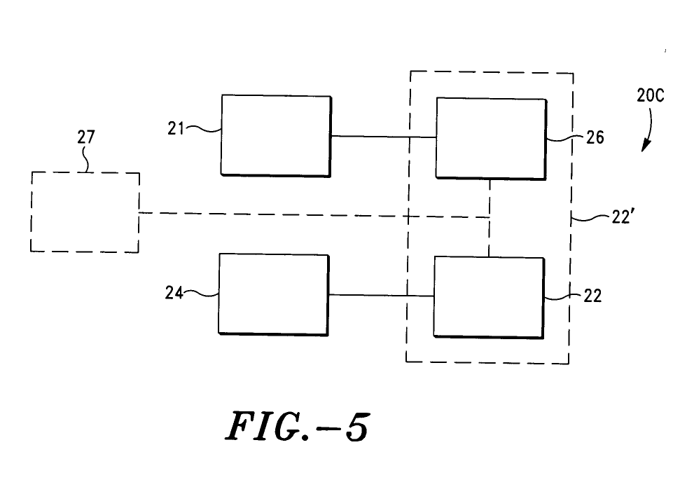

Referring now to FIG. 5, there is shown yet another embodiment of a control system 20C of the invention. As illustrated in FIG. 5, the control system 20C similarly includes a control module 22 and a treatment member 24. The system 20C further includes at least one signal sensor 21.

The system 20C also includes a processing module (or computer) 26. According to the invention, the processing module 26 can be a separate component or a sub-system of a control module 22′, as shown in phantom.

The processing module 26 preferably includes storage means adapted to store the captured neuro-electrical signals that produce a satiety effect in the body. In a preferred embodiment, the processing module 26 is further adapted to extract and store the components of the captured neuro-electrical signals in the storage means according to the function performed by the signal components.

In one embodiment of the invention, the control system 20C also includes a remote transponder 27 that is adapted to transmit controls signals to the module 22′ and/or processing module 26. In another embodiment, the transponder 27 is further adapted to monitor the control parameters of the module 22′ and/or processing module 26.

According to the invention, the food intake control systems 20A, 20B, 20C, described above, can be employed as stand-alone systems or employed in conjunction with (or as an integral component or sub-system of) another gastric device, such as a gastric band.

As will be readily apparent to one having ordinary skill in the art, the food intake control systems 20A, 20B, 20C can be employed in conjunction with or as an integral component (or sub-system) of various gastric bands. Illustrative are the gastric bands disclosed in U.S. Pat. No. 5,601,604, U.S. application Ser. Nos. 11/118,452 (Pub. No. 2006/0247719), 11/296,258 (Pub. No. 2006/0129027) and 11/118,980 (Pub. No. 2006/0247722) and EP Application Nos. 1205148 and 1547549.

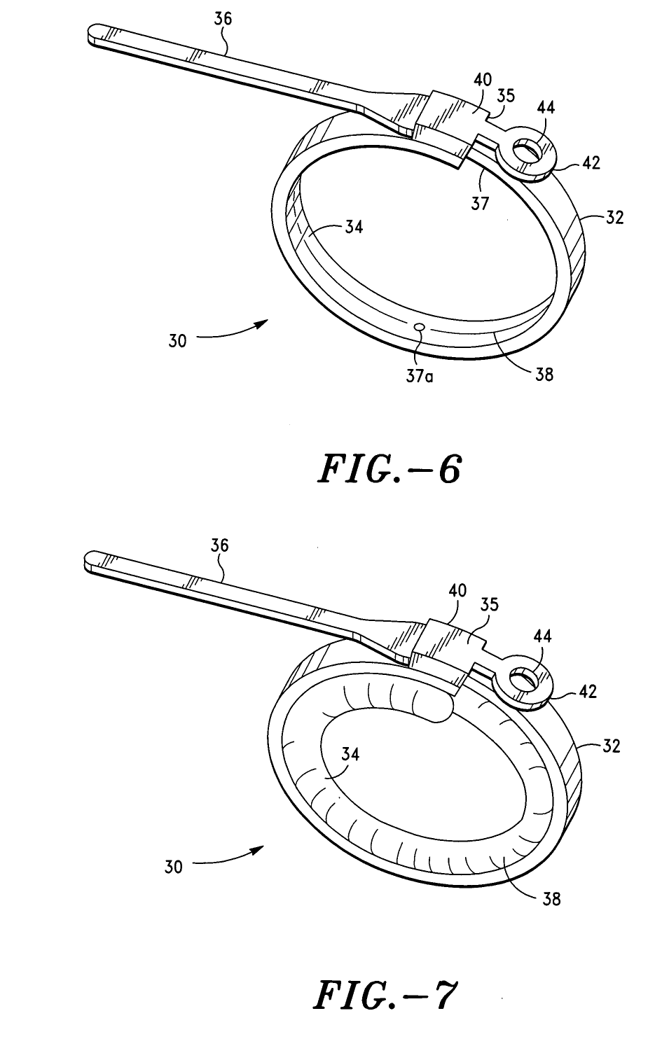

Referring now to FIG. 6, there is shown one embodiment of a suitable gastric band that can be employed within the scope of the invention. As illustrated in FIG. 6, the gastric band (designated generally “30”) includes a body portion 32 having an inner stomach facing surface 34. The body portion 32 includes a head end 35 and a tail end 37 having one or more suture holes therein.

The gastric band 30 further includes a fill tube 36 (having a lumen therein) that is in fluid communication with an inflatable member 38, which is disposed on the inner surface 34. The inflatable member 38 includes a lumen opening 37a that is adapted to receive the fill tube lumen.

The head end 35 of the body portion 32 includes a buckle 40. The buckle 40 includes a pull tab 42 having a suture hole 44 integral therewith.

In practice, the gastric band 30 is placed in a circling position around the stomach 13; preferably, the neck region of the stomach 13. This is typically accomplished by pushing the fill tube 36 through a laparoscopic canula (not shown) in the patient's abdomen. The end of the fill tube 36 is passed around the stomach 13, and the tail 37 is attached to the buckle 40, so that the buckle 40 and the tail 37 are irreversibly affixed to one another. The adjustment of the stoma; the narrow opening in the stomach created by the band, is calibrated by a second step after the band 30 is secured in this single position.

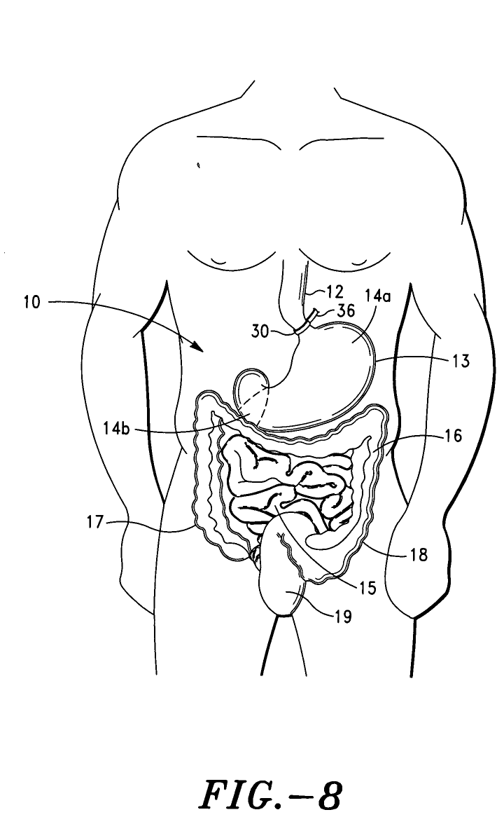

The interior of the inflatable member 38 is in fluid communication with an injection reservoir (not shown) by means of the fill tube 36. In practice, the inflatable member 38 is gradually filled with saline, whereby the inflatable member 38 expands (see FIG. 7), and, as illustrated in FIG. 8, presses on and contracts the upper portion of the stomach wall underlying the band 30. This results in the decrease of the opening (stoma) inside the stomach 13 directly under the encircling band 30.

Referring now to FIG. 9, there is shown one embodiment of a gastric restriction system of the invention. As illustrated in FIG. 9, the gastric restriction system (designated generally “50A”) includes the gastric band 30 and control system 20B, discussed above.

As indicated, any of the aforementioned food intake control systems, i.e. 20A, 20B, 20C, and equivalents thereof, can be employed with the gastric restriction system 50A illustrated in FIG. 9. The illustrated system 50A should thus not be construed as limiting the scope of the invention in any manner.

According to the invention, the transmitter of the gastric restriction system 50A comprises an electrode system, having at least two (2) electrodes. Referring to FIG. 9, in the illustrated embodiment, the electrode system (designated generally “25”) includes two electrodes 25a, 25b that are disposed concentrically, i.e. longitudinally, on the exterior surface of the inflatable member 38.

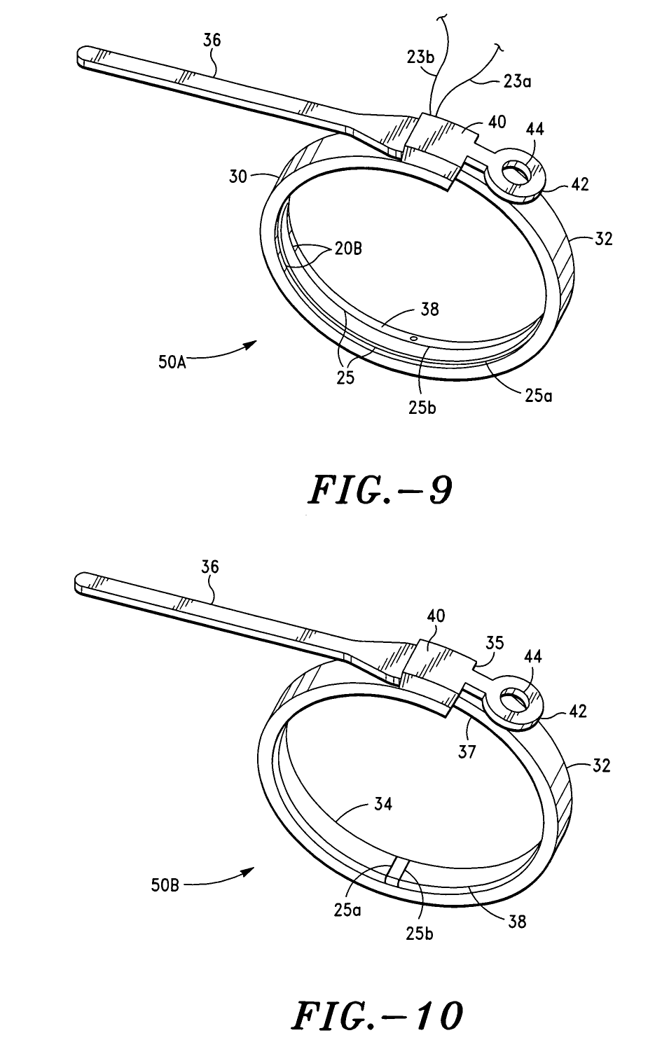

Referring now to FIG. 10, in another embodiment of the invention, the gastric restriction system (designated “50B”) similarly includes electrodes 25a, 25b. As illustrated in FIG. 10, the electrodes 25a, 25b are, however, disposed in a substantially angular or perpendicular orientation on the inflatable member 38.

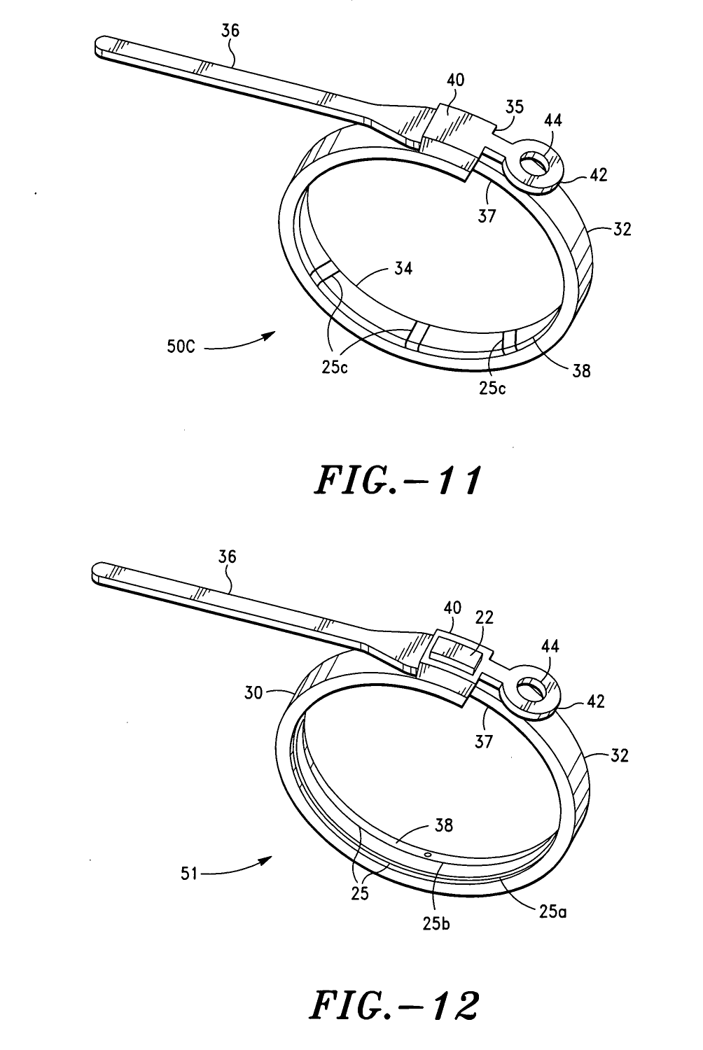

According to the invention, the electrode system 25 can also comprise a band of multiple electrodes, i.e. multiple pairs of electrodes. The band of multiple electrodes can similarly be disposed concentrically, or substantially angularly or perpendicular on the inflatable member 38 (see system 50C in FIG. 11).

Referring back to the embodiment shown in FIG. 9, the electrode system 50 includes two interconnect wires 23a, 23b that are in communication with the electrodes 25a, 25b and the control module (not shown).

According to the invention, the electrodes 25a, 25b can comprise any suitable biocompatible, conductive material, such as platinum foil and platinum-iridium. Similarly, the electrode interconnect wires, i.e. 23 (see FIG. 4) and 23a, 23b (see FIG. 9), can be fabricated from any suitable biocompatible, conductive, and, preferably, low fatigue material, such as platinum-iridium.

According to the invention, the control module can be disposed external of the body or implanted in the subject, e.g., secured to a wall of the stomach 13. Thus, in some embodiments of the invention, the control module 22 is disposed on the gastric band 30 or an integral component thereof. Illustrative is the embodiment shown in FIG. 12, wherein the control module 22 is disposed on the buckle 40 of the gastric band 30.

According to the invention, if the control module is implanted in the subject or disposed on or in the gastric band, the gastric system 50 would include a remote transponder 27, discussed above, to facilitate communications by and between the subject (and/or medical practitioner) and the control module.

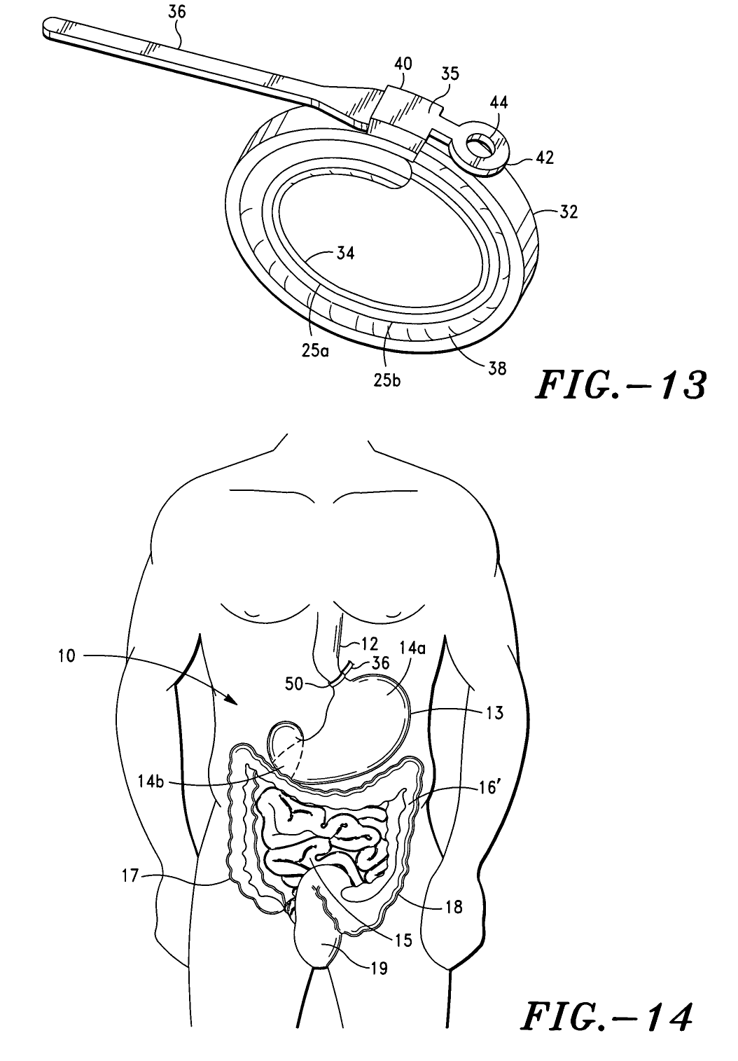

Operation of the gastric restriction system 50 will now be discussed in detail. In accordance with one embodiment of the invention, the gastric restriction system 50 is initially positioned on the neck of the stomach. The inflatable member 38 is then gradually filled with a predetermined amount of saline, whereby the inflatable member 38 expands (see FIG. 13), places the electrodes 25a, 25b in intimate contact with the neck of the stomach, and constricts the stoma of the stomach (see FIG. 14), during which time one or more synthesized satiety signals are transmitted to the subject.

As illustrated in FIG. 14, the synthesized satiety signals are transmitted proximate the positioned system 50. As set forth above, the synthesized satiety signals can be transmitted at pre-programmed set intervals and/or manually.

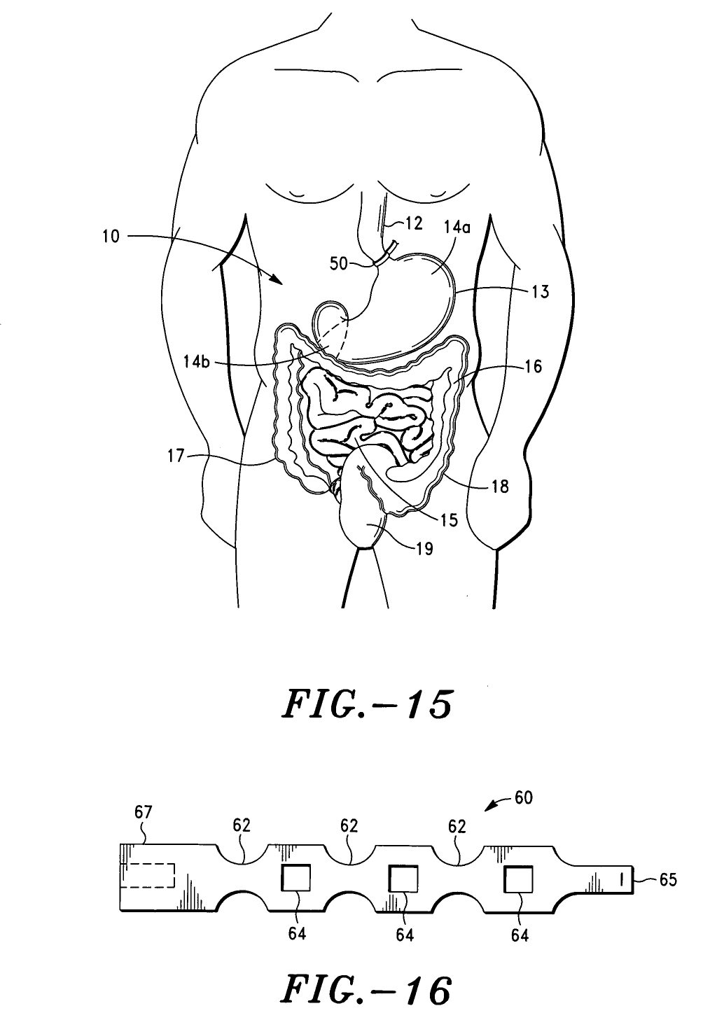

According to the invention, the gastric restriction system 50 can also effectively transmit synthesized satiety signals to the subject without inflating the inflatable member 38 and, hence, constricting the stoma of the stomach. As will be apparent to one having ordinary skill in the art, this can be achieved by “non-restrictively” positioning the gastric restriction system 50 on the neck of the stomach 13, whereby the electrodes 25a, 25b are placed in intimate contact with the neck of the stomach 13 (see FIG. 15).

According to the invention, various other non-restrictive electrode positioning techniques and devices can also be employed to position the electrodes at desired locations proximate the stomach.

Referring now to FIG. 16, there is shown one embodiment of a gastric electrode positioning device (designated generally “60”). As illustrated in FIG. 16, the positioning device 60 comprises a thin, elongated band having an engagement end 65 and a securing end 67. The securing end 67 preferably includes internal means for receiving and securing the engagement end 65.

The positioning device 60 further includes at least one, preferably, a plurality of highly elastic regions 62 that facilitate expansion of the device 60 while disposed on the neck of the stomach 13. As will be appreciated by one having ordinary skill in the art, the highly elastic regions can be provided by various means. For example, the elastic regions can comprise regions of reduced area, thinner cross-section, corrugated regions, etc., and combinations thereof.

Thus, in one embodiment, as illustrated in FIG. 16, the highly elastic regions 62 comprise regions of reduced area. In other envisioned embodiments, the highly elastic regions comprise regions having thinner cross-sections. In other embodiments, the highly elastic regions comprise corrugated regions.

According to the invention, the electrodes can be disposed on the positioning device 60 in any operable location, whereby the electrodes are in contact with the neck of the stomach 13 when the positioning device is non-restrictively positioned thereon.

Referring back to FIG. 16, the illustrated positioning device 60 includes multiple electrodes 64. As illustrated in FIG. 16, the electrodes 64 are disposed in spaced regions of the device 60 between the highly elastic regions 62.

In accordance with one embodiment of the invention, the method for treating eating disorders thus includes the steps of (i) generating a synthesized neurosignal that substantially corresponds to a neuro-electrical signal that is generated in a body and produces a satiety effect in the body and (ii) transmitting the synthesized satiety signal to the subject. Preferably, the synthesized neurosignal comprises a synthesized satiety signal.

In another embodiment of the invention, the method for treating eating disorders includes the step of constricting the stoma of the stomach.

In one embodiment, the synthesized neurosignal is transmitted at predetermined time intervals.

In one embodiment, the synthesized neurosignal is transmitted manually.

In another embodiment, the synthesized neurosignal is transmitted manually and at predetermined time intervals.

In one embodiment of the invention, the synthesized neurosignal has a first region having a first positive voltage in the range of approximately 100-1500 mV for a first period of time in the range of approximately 100-400 μsec and a second region having a first negative voltage in the range of approximately −50 mV to −750 mV for a second period of time in the range of approximately 200-800 μsec.

In a preferred embodiment of the invention, the first positive voltage is approximately 800 mV, the first period of time is approximately 200 μsec, the first negative voltage is approximately −400 mV and the second period of time is approximately 400 μsec.

Preferably, the synthesized neurosignal has a repetition rate in the range of approximately 0.01-4 KHz.

In another embodiment of the invention, the method for treating eating disorders includes the steps of (i) capturing neuro-electrical signals that are generated in the body and produce a satiety effect in the body, (ii) generating a synthesized neurosignal that substantially corresponds to at least one of the captured neuro-electrical signals, and (iii) transmitting the synthesized neurosignal to the subject. Preferably, the synthesized neurosignal comprises a synthesized satiety signal.

In another embodiment of the invention, the method for treating eating disorders includes the step of constricting the stoma of the stomach.

According to the invention, a single synthesized neurosignal or a plurality of synthesized neurosignals can be transmitted to the subject in conjunction with one another.

The system for treating eating disorders, in accordance with one embodiment of the invention, generally comprises (i) a processor adapted to generate at least a first synthesized neurosignal that substantially corresponds to a neuro-electrical signal that is generated in the body and produces a satiety effect in the body, and (ii) a signal transmitter adapted to be in communication with the subject's body for transmitting the first synthesized neurosignal to the subject.

In another embodiment of the invention, the system includes a gastric band that is adapted to constrict the stoma of the stomach.

In another embodiment of the invention, the system includes a signal sensor that is adapted to be in communication with the subject's body for sensing neuro-electrical signals that are generated in the body and transmitting the sensed signals to the processor.

In another embodiment, the system includes a remote transponder that is adapted to transmit control signals to the processor.

In another embodiment, the remote transponder is further adapted to remotely monitor the control parameters of the processor.

### EXAMPLES

Methods of using the methods and systems of the invention will now be described in detail. The methods set forth herein are merely examples of envisioned uses of the methods and systems to control and/or limit food intake and thus should not be considered as limiting the scope of the invention.

### Example 1

A 45 year old female suffers from morbid obesity. She has been overweight since a first pregnancy, and her weight is now in excess of 200 percent of her ideal weight. She suffers from hypertension and sleep apnea, which her physician believes are directly related to her weight problem.

The patient consults with a physician and dietician to work out a diet and walking regimen for long-term weight loss. In coordination with this regimen, the patient has a gastric restriction system, such as system 50 (discussed above), implanted in her body. In this example, the gastric restriction system is designed to generate and transmit synthesized neurosignals that correspond to neuro-electrical signals that derive from the neck of the stomach that elicit a feeling of fullness or satiety in the brain.

In this example, the patient monitors her weight weekly. It is expected that the patient will have periodic visits to her primary care physician for adjustment in the gastric band, if necessary, and timing and duration of the synthesized signals. It is also anticipated that the patient will remain on the exercise and diet regimen during treatment.

As will be appreciated by one having ordinary skill in the art, the present invention provides numerous advantages. Among the advantages are the provision of a method and system for treating eating disorders having:

Enhanced effectiveness;
Reduced deleterious side effects;
More effective satiety effects; and
Less user discomfort.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for treating eating disorders, comprising the steps of:
generating a neuro-electrical satiety signal that produces a satiety effect in the body, said neuro-electrical satiety signal having a positive voltage region having a positive voltage in the range of approximately 100-1500 mV for a first period of time in the range of approximately 100-400 μsec and a negative region having a negative voltage in the range of approximately −50 mV to −750 mV for a second period of time in the range of approximately 200-800 μsec; and
transmitting said neuro-electrical satiety signal to a subject's body, whereby a satiety effect is produced therein.

2. A method for treating eating disorders, comprising the steps of:
generating a synthesized neurosignal that substantially corresponds to a neuro-electrical signal that is generated in a body and produces a satiety effect in the body;
constricting the stoma of the stomach; and
transmitting said synthesized neurosignal to a subject's body, whereby a satiety effect is produced therein.

3. The method of claim 2, wherein said synthesized neurosignal has a positive voltage region having a positive voltage in the range of approximately 100-1500 mV for a first period of time in the range of approximately 100-400 μsec and a negative region having a negative voltage in the range of approximately −50 mV to −750 mV for a second period of time in the range of approximately 200-800 μsec.

4. The method of claim 3, wherein said synthesized neurosignal has a frequency in the range of approximately 0.5-4 KHz.

5. The method of claim 2, wherein a plurality of said synthesized neurosignals is generated and transmitted to said subject.

6. A system for treating eating disorders, comprising:
a gastric band adapted to constrict the stoma of a subject's stomach;
a processor adapted to generate at least a first synthesized neurosignal, said first synthesized neurosignal substantially corresponding to a neuro-electrical signal that is generated in the body and produces a satiety effect in the body; and
a signal transmitter adapted to be in communication with said subject's body for transmitting said first synthesized neurosignal to said subject.

7. The system of claim 6, wherein said first synthesized neurosignal has a positive voltage region having a positive voltage in the range of approximately 100-1500 mV for a first period of time in the range of approximately 100-400 μsec and a negative region having a negative voltage in the range of approximately −50 mV to −750 mV for a second period of time in the range of approximately 200-800 μsec.

8. The system of claim 7, wherein said first synthesized neurosignal has a frequency in the range of approximately 0.5-4 KHz.

9. A system for treating eating disorders, comprising:
a gastric band adapted to constrict the stoma of a subject's stomach;
at least one sensor adapted to sense at least a first neuro-electrical signal that is generated in the body and produce a satiety effect in the body, said sensor being further adapted to generate and transmit at least one sensor signal corresponding to said first neuro-electrical signal;
a processor adapted to receive said sensor signal, said processor being further adapted to generate a first synthesized neurosignal, said first synthesized neurosignal substantially corresponding to said first neuro-electrical signal;
a transponder adapted to transmit control signals to said processor; and
a signal transmitter adapted to be in communication with the subject's body for transmitting said first synthesized neurosignal to said subject.
